(19) United States
(12) Patent Application Publication
(10) Pub. No.: US 2025/0370078 A1
(43) Pub. Date: Dec. 4, 2025

(54) SYSTEMS AND METHODS FOR MYOCARDIAL STRAIN ANALYSIS USING MAGNETIC RESONANCE IMAGING

(71) Applicant: The Curators of the University Of Missouri, Columbia, MO (US)

(72) Inventors: Changyu Sun, Columbia, MO (US); Talissa Ann Altes, Columbia, MO (US)

(21) Appl. No.: 19/213,211

(22) Filed: May 20, 2025

Related U.S. Application Data
(60) Provisional application No. 63/653,334, filed on May 30, 2024.

(51) Int. Cl. G01R 33/56; A61B 5/00; A61B 5/055; G01R 33/48; G01R 33/567; G06T 7/00; G06T 7/20

(52) U.S. Cl. CPC G01R 33/5608; A61B 5/0044; A61B 5/055; G01R 33/4818; G01R 33/567; G06T 7/0012; G06T 7/20; G06T 2207/10088; G06T 2207/20016; G06T 2207/20056; G06T 2207/20081; G06T 2207/20084; G06T 2207/30048

(57) ABSTRACT

A method for analyzing myocardial strain in a subject using magnetic resonance imaging (MRI) is provided. The method includes acquiring cine images and low-resolution tagging images of a cardiac region of a subject within a single breath-hold, the cine images having a first resolution, the tagging images having a second resolution lower than the first resolution. The method also includes deriving high-resolution tagging images based on the cine images and the low-resolution tagging images, the high-resolution tagging images having a resolution higher than the second resolution. The method also includes estimating intramyocardial motion based on the high-resolution tagging images and/or the cine images. The method also includes generating myocardial strain maps based on the intramyocardial motion. The method further includes outputting the myocardial strain maps.

### CROSS-REFERENCE TO RELATED APPLICATIONS

This patent application claims the benefit of U.S. Provisional Patent Application No. 63/653,334, filed on May 30, 2024, titled “SYSTEMS AND METHODS FOR MYOCARDIAL STRAIN ANALYSIS USING MAGNETIC RESONANCE IMAGING,” the entire content and disclosures of which is hereby incorporated herein by reference in their entirety.

### BACKGROUND

The field of the disclosure relates generally to medical systems and methods, and more particularly, to systems and methods medical systems and methods for strain analysis.

Magnetic resonance imaging (MRI) has proven useful in diagnosis of many diseases. MRI provides detailed images of soft tissues, abnormal tissues such as tumors, and other structures, which cannot be readily imaged by other imaging modalities, such as computed tomography (CT). Further, MRI operates without exposing patients to ionizing radiation experienced in modalities such as CT and x-rays.

Cardiac strain assessments require additional images for physical tissue property measurement. Known methods are disadvantageous in some aspects and improvements are desired.

### BRIEF DESCRIPTION

In one aspect, a method for analyzing myocardial strain in a subject using magnetic resonance imaging (MRI) is provided. The method includes acquiring, via a magnetic resonance (MR) system, cine images and low-resolution tagging images of a cardiac region of a subject within a single breath-hold of the subject, the cine images having a first resolution, the tagging images having a second resolution lower than the first resolution. The method also includes deriving high-resolution tagging images based on the cine images and the low-resolution tagging images, the high-resolution tagging images having a resolution higher than the second resolution. The method also includes estimating intramyocardial motion based on the high-resolution tagging images and/or the cine images. The method also includes generating myocardial strain maps based on the intramyocardial motion. The method further includes outputting the myocardial strain maps.

The method may include deriving the high-resolution tagging images using a first neural network model, wherein the first neural network model is trained with a pair of pristine images and crude images, wherein the pristine images are the crude images with noise reduced and/or having a resolution higher than a resolution of the crude images, and the target output images of the first neural network model are the pristine images.

The method may also include estimating the intramyocardial motion using a second neural network model, wherein the second neural network model is trained via unsupervised training with pairs of training cine images and training high-resolution tagging images, the training high-resolution tagging images having a resolution higher than the second resolution, wherein the pairs of the training cine images and the training high-resolution tagging images are input into the second neural network model during the unsupervised training.

The method may further include estimating the intramyocardial motion by inputting magnitude images and/or phase images of the high-resolution tagging images and/or magnitude images of the cine images into a third neural network model, the third neural network model having a plurality of input channels.

The method may further include generating the myocardial strain maps by deriving pseudo-balanced steady state free precession (bSSFP) cine images based on the cine images and/or the high-resolution tagging images, the pseudo-bSSFP cine images having a contrast resembling a contrast of images acquired by a bSSFP MR pulse sequence.

The method may also include generating masks based on the pseudo-bSSFP cine images, and generating the myocardial strain maps by generating the myocardial strain maps by applying generated masks to images of the cine images and/or the high-resolution tagging images to generate myocardial contours, and generating the myocardial strain maps based on the intramyocardial motion and the myocardial contours.

In another aspect, a computer-implemented method for analyzing myocardial strain in a subject using MRI is provided. The method includes receiving low-resolution tagging images of a cardiac region of a subject, the low-resolution tagging images acquired via a magnetic resonance system within a single breath-hold of the subject. The method also includes deriving high-resolution tagging images based on the low-resolution tagging images, the high-resolution tagging images having a resolution higher than the low-resolution tagging images. The method further includes estimating intramyocardial motion based on the high-resolution tagging images, generating myocardial strain maps based on the intramyocardial motion, and outputting the myocardial strain maps.

### DRAWINGS

FIG. 1 is a flow chart of an example method of automatic strain analysis.

FIG. 2 is a schematic diagram of an example magnetic resonance imaging (MRI) system machine in accordance with an embodiment of the present invention.

FIG. 3A is a schematic diagram of a neural network model.

FIG. 3B is a schematic diagram of a neuron in the neural network model shown in FIG. 3A.

FIG. 4 is a block diagram of an example computing device.

FIG. 5 is a block diagram of an example server computing device.

FIG. 6 is a flow chart depicting the CineTag imaging process. A Standard cine MRI (A) and the paired low resolution (LR) tagging image (B) are simultaneously acquired from the LR tagging calibrated cine pulse sequence. The LR tagging is enhanced to high resolution (HR) tagging (C) using the super-resolution (SR) network (Tag-SR). The HR tagging is then analyzed alongside paired cine images by the CineTag-Motion to calculate per-pixel displacement (F). Concurrently, cine images undergo well-established automatic cine balanced steady state free precession (bSSFP) segmentation, delineating the endocardial and epicardial borders. These contours are directly transferred to the matched HR Tagging (E) and to the displacement map, facilitating the consequent intramyocardial strain analysis (G).

FIG. 7 illustrates the design for the neural network to provide enhanced resolution tagging. Low-resolution LR tagging images are reconstructed from undersampled k-space by inverse Fourier Transform. Enhanced-resolution tagging will be generated from a diffusion model with low-resolution tagging as the input.

FIG. 8 shows example short-axis and 2-chamber long-axis MR tagging images of a heart failure pig with myocardial infarction at septal and anterior segments. Enhanced super-resolution images were produced by applying a known method of ESRGAN (B, F) and the Tag-SR model (C, G) to the synthesized low-resolution tagging images (A, E). The Tag-SR model generated clearer tag lines and showed better agreement with the high-resolution images than ESRGAN.

FIG. 9 illustrates an embodiment of the CineTag-Motion network. Paired multiphase tagging and cine images are processed by the network to acquire both Euler and Lagrangian Displacement, which will then be wrapped with the reference frame to get moved images. Smoothness loss and similarity loss will be calculated to update the model.

FIG. 10 shows the MR tagging and the displacement map corresponding to the LGE measurements machine in accordance with an embodiment of the present invention. Example end-diastolic (A) and end-systolic (B) MR tagging images of a heart failure pig with myocardial infarction. The application of a preliminary Tag-Motion model to this multiphase tagging images provided 2D displacement maps, as exemplified by the end-systolic phase in (C), which demonstrated diminished wall motion at septal and anterior segments and normal strain elsewhere. Corresponding LGE MRI image (D) shows a transmural infarct at the dyskinetic region.

FIG. 11 illustrates the design of the IMPACTNet. Network inputs are 3-channel 2D+t images including a series of magnitude images and two harmonic phases for two displacement directions. Both Euler and Lagrangian displacements will be estimated and wrapped with the reference frame to get moved images. Smoothness loss and similarity loss will be calculated to update the model.

FIG. 12 illustrates an embodiment of the standardized automatic cardiac MR Image segmentation using generative AI. During training, the model will be trained with text-image pair to learn from random noise and map into cardiac magnetic resonance (CMR) image domains including cine, tagging, perfusion, LGE, T1 mapping and so on. During inference, the targeted sequence will serve as the anatomy condition and be diffused into the Mth step during the diffusion process. The segmentation from the generated intermediate cine will be applied to the original anatomy image.

FIG. 13 shows an example cine image and an example tagging image.

FIG. 14 shows an example pulse sequence for acquiring cine images and tagging images.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

The disclosure includes systems and methods of strain measurement using magnetic resonance (MR) imaging (MRI) of a subject. As used herein, a subject is a human, an animal, or a phantom, or part of a human, an animal, or a phantom, such as an organ or tissue. Noise, artifacts, other undesired signals, or any combination thereof is collectively referred to as noise. Artifacts may be caused by under-sampling, eddy currents, physiological noise, B0 drifts, B0 and/or B1 inhomogeneity, or other system imperfections. Compared to a crude image, a pristine image is an image with noise reduced or resolution increased. For example, a crude image may be under-sampled, and/or have a lower resolution than the pristine image. Reducing or removing noise is collectively referred to as reducing noise or denoising. A denoised image is an image having noise reduced. Method aspects will be in part apparent and in part explicitly discussed in the following description.

Myocardial strain, or the change in myocardial fiber length over the cardiac cycle, is a measure of cardiac muscle function. Cardiac strain is typically measured using conventional techniques such as echocardiography and magnetic resonance imaging, adding additional clinical information to augment the current techniques. Myocardial strain identifies global and regional abnormalities in myocardial function and differentiates types of cardiomyopathy. It is an earlier marker of myocardial disease than ejection fraction and is predictive of cardiovascular adverse events. Accurate measurement requires high-quality images and experienced practitioners.

Feature tracking (FT) is a post-processing technique applied to standard cine cardiac MRI images, which enables the measurement of longitudinal, circumferential, and radial strain without necessitating additional imaging time and resources. For example, FT includes identifying end-diastole and end-systole, detecting endocardial and epicardial borders either semiautomatic or by manual contouring (papillary muscles are typically excluded from the endocardial contour), defining the segment of the MRI image to be tracked, and calculating, using an algorithm, measurements for features such as longitudinal, circumferential, and radial strain, as well as ejection fraction. Accordingly, feature tracking provides an efficient and easy-to-use process for cardiac strain measurements resulting from its ability to leverage existing MRI data. However, its reliance on a two-dimensional contour-based tracking algorithm introduces significant drawbacks, such as low accuracy due to lack of features to be tracked within the myocardium region in cine MR images. Moreover, feature tracking's inability to accurately measure segmental and pixel-wise strain restricts its utility for comprehensive myocardial motion assessments.

Additionally, strain-dedicated sequences (e.g. tagging) is used as a reference standard for myocardial strain imaging. Tagging is an MRI technique where RF pulses are used to place stripes or grids on the heart to follow the motion of the heart during the cardiac cycle. This technique creates visible patterns superimposed on the myocardium, such as parallel lines or grids, which serve as markers for tracking tissue displacement during cardiac cycles. Tagging may include image preparation, endocardial and epicardial border detection, myocardial region definition for tracking, tracking the tag, tracking of dark lines or intersections, performing harmonic phase analysis (HARP), performing other optical flow techniques, getting estimated motion, and performing strain calculation.

Accordingly, tagging provides a direct measurement of physical tissue properties from the MRI images. Tagging provides extensively validated strain assessments from the measurements. Tagging may be limited by low spatial resolution. Additionally, tagging methods experience delayed tag deposition at the onset of systole leading to relatively low accuracy in strain assessment. Tagging experiences reduced accuracy in areas of thin myocardial walls, and the fading of tags throughout the cardiac cycle. Additionally, tagging requires additional images for strain analysis, increasing the demand on MRI resources. Tagging also requires significant computing resources for elaborate post-processing to determine motion quantification. These conventional solutions further increases evaluation times required that cannot be automated.

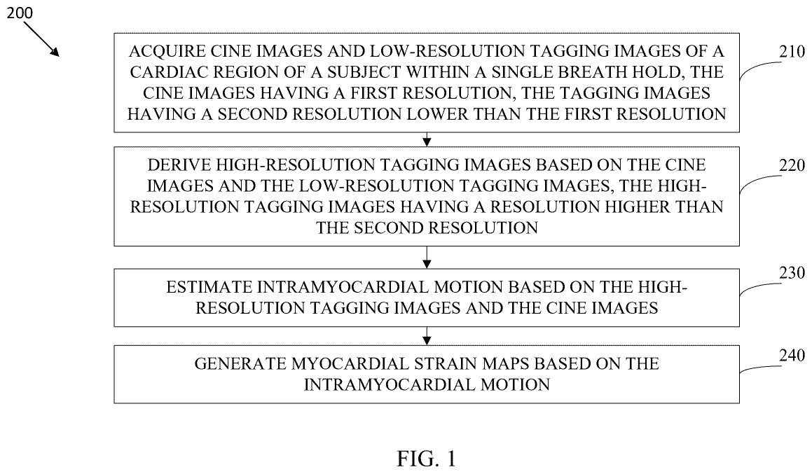

FIG. 1 is a flow chart of an example method 200 of strain analysis. In the example embodiment, method 200 may be implemented on an MRI system or a computing device in communication with an MR system. In the example embodiment, method 200 includes acquiring 210 cine images and low-resolution (LR) tagging images of a cardiac region of a subject. The images of the subject may be acquired within a single breath-hold. The cine images and the tagging images are different resolution images. For example, the cine images include a higher resolution than the tagging images.

In the example embodiment, method 200 further includes deriving 220 high resolution (HR) tagging images based on the cine images and the LR tagging images. The HR tagging images have a higher resolution than the LR tagging images. In some embodiments, the HR tagging images are derived using a first neural network model. The first neural network model may be trained with a pair of pristine images and crude images. The pristine images have reduced noise and higher resolution than the crude images. The target output of the first neural network model are the pristine images.

In the example embodiment, method 200 further includes estimating 230 the intramyocardial motion based on the HR tagging images and/or the cine images. In some embodiments, the intramyocardial motion is estimated using a second neural network model. The second neural network model is trained via unsupervised training with pairs of training cine images and training high-resolution tagging images. The training high-resolution images have a higher resolution than the acquired tagging images. The pairs of training cine images and training high-resolution tagging images are input into the second neural network model during the unsupervised training. In other embodiments, a third neural network model is used for estimating 230 the intramyocardial motion. The third neural network model includes a plurality of input channels. The third neural network model receives inputs of magnitude image and phase images of the HR tagging images and/or magnitude images of the cine images.

In the example embodiment, method 200 further includes generating 240 myocardial strain maps based on the intramyocardial motion. In some embodiments, method 200 includes deriving pseudo-balanced steady state free precession (bSSFP) cine images based on images acquired by i) MR tagging sequences, ii) other non-cine sequences such as late gadolinium enhancement (LGE), T1 mapping, T1 rho, T2 mapping, or perfusion, or iii) non-bSSFP cine sequences such as gradient-echo (GRE) cine sequence, and/or crude bSSFP cine images such as bSSFP cine images having artifacts like banding artifacts and/or breathing artifacts. The pseudo-bSSFP cine images have a contrast resembling a contrast of images acquired by a bSSFP MR pulse sequence. As used herein, a contrast of an image resembling a contrast of another image refers to that the contrasts between the two images are the same or the differences of the contrasts are at or below a level. In various embodiments, method 200 also includes generating masks based on the pseudo-bSSFP cine images and segmenting images of the intramyocardial motion and the HR tagging images with the generated masks.

In some embodiments, cine images paired with the tagging images are unavailable. The motion may be estimated based on the high-resolution tagging images alone.

In the example embodiment, method 200 further includes outputting the myocardial strain maps. For example, the myocardial strain maps may be output to a display or other device. The myocardial strain maps may be used to diagnose cardiac diseases.

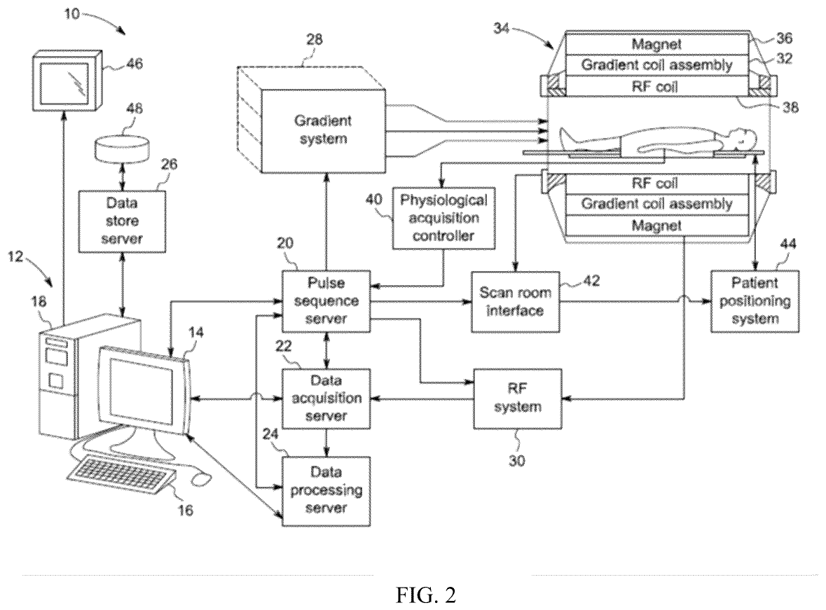

FIG. 2 illustrates a schematic diagram of an example MRI system 10. In magnetic resonance imaging (MRI), a subject is placed in a magnet. When the subject is in the magnetic field generated by the magnet, magnetic moments of nuclei, such as protons, attempt to align with the magnetic field but precess about the magnetic field in a random order at the nuclei's Larmor frequency. The magnetic field of the magnet is referred to as B0 and extends in the longitudinal or z direction. In acquiring an MRI image, a magnetic field (referred to as an excitation field B1), which is in the x-y plane and near the Larmor frequency, is generated by a radio-frequency (RF) coil and may be used to rotate, or “tip,” the net magnetic moment Mz of the nuclei from the z direction to the transverse or x-y plane. A signal, which is referred to as an MR signal, is emitted by the nuclei, after the excitation signal B1 is terminated. To use the MR signals to generate an image of a subject, magnetic field gradient pulses (Gx, Gy, and Gz) are used. The gradient pulses are used to scan through the k-space, the space of spatial frequencies or inverse of distances. A Fourier relationship exists between the acquired MR signals and an image of the subject, and therefore the image of the subject may be derived by reconstructing the MR signals.

In the example embodiment, MRI system 10 includes a workstation 12 having a display 14 and a keyboard 16. Workstation 12 includes a processor 18, such as a commercially available programmable machine running a commercially available operating system. Workstation 12 provides an operator interface that allows scan prescriptions to be entered into MRI system 10. Workstation 12 is coupled to a pulse sequence server 20, a data acquisition server 22, a data processing server 24, and a data store server 26. Workstation 12 and each server 20, 22, 24, and 26 communicate with each other.

In the example embodiment, pulse sequence server 20 responds to instructions downloaded from workstation 12 to operate a gradient system 28 and a radiofrequency (“RF”) system 30. The instructions are used to produce gradient and RF waveforms in MR pulse sequences. An RF coil 38 and a gradient coil assembly 32 are used to perform the prescribed MR pulse sequence. RF coil 38 is shown as a whole body RF coil. RF coil 38 may also be a local coil that may be placed in proximity to the anatomy to be imaged, or a coil array that includes a plurality of coils.

In the example embodiment, gradient waveforms used to perform the prescribed scan are produced and applied to gradient system 28, which excites gradient coils in gradient coil assembly 32 to produce the magnetic field gradients Gx, Gy, and Gz used for position-encoding MR signals. Gradient coil assembly 32 forms part of a magnet assembly 34 that also includes a polarizing magnet 36 and RF coil 38.

In the example embodiment, RF system 30 includes an RF transmitter for producing RF pulses used in MR pulse sequences. The RF transmitter is responsive to the scan prescription and direction from pulse sequence server 20 to produce RF pulses of a desired frequency, phase, and pulse amplitude waveform. The generated RF pulses may be applied to RF coil 38 by RF system 30. Responsive MR signals detected by RF coil 38 are received by RF system 30, amplified, demodulated, filtered, and digitized under direction of commands produced by pulse sequence server 20. RF coil 38 is described as a transmitter and receiver coil such that RF coil 38 transmits RF pulses and detects MR signals. In one embodiment, MRI system 10 may include a transmitter RF coil that transmits RF pulses and a separate receiver coil that detects MR signals. A transmission channel of RF system 30 may be connected to a RF transmission coil and a receiver channel may be connected to a separate RF receiver coil. Often, the transmission channel is connected to the whole body RF coil 38 and each receiver section is connected to a separate local RF coil.

In the example embodiment, RF system 30 also includes one or more RF receiver channels. Each RF receiver channel includes an RF amplifier that amplifies the MR signal received by RF coil 38 to which the channel is connected, and a detector that detects and digitizes the I and Q quadrature components of the received MR signal. The magnitude of the received MR signal may then be determined as the square root of the sum of the squares of the I and Q components as in Eq. (1) below:

$$M = \sqrt{{I}^{2} + {Q}^{2}} ; \qquad ( 1 )$$

and the phase of the received MR signal may also be determined as in Eq. (2) below:

$$φ = {tan}^{- 1} ( \frac{Q}{I} ) . \qquad ( 2 )$$

In the example embodiment, the digitized MR signal samples produced by RF system 30 are received by data acquisition server 22. Data acquisition server 22 may operate in response to instructions downloaded from workstation 12 to receive real-time MR data and provide buffer storage such that no data is lost by data overrun. In some scans, data acquisition server 22 does little more than pass the acquired MR data to data processing server 24. In scans that need information derived from acquired MR data to control further performance of the scan, however, data acquisition server 22 is programmed to produce the needed information and convey it to pulse sequence server 20. For example, during prescans, MR data is acquired and used to calibrate the pulse sequence performed by pulse sequence server 20. Also, navigator signals may be acquired during a scan and used to adjust the operating parameters of RF system 30 or gradient system 28, or to control the view order in which k-space is sampled.

In the example embodiment, data processing server 24 receives MR data from data acquisition server 22 and processes it in accordance with instructions downloaded from workstation 12. Such processing may include, for example, Fourier transformation of raw k-space MR data to produce two or three-dimensional images, the application of filters to a reconstructed image, the performance of a back projection image reconstruction of acquired MR data, the generation of functional MR images, and the calculation of motion or flow images.

In the example embodiment, images reconstructed by data processing server 24 are conveyed back to, and stored at, workstation 12. In some embodiments, real-time images are stored in a database memory cache (not shown in FIG. 1), from which they may be output to operator display 14 or a display 46 that is located near magnet assembly 34 for use by attending physicians. Batch mode images or selected real time images may be stored in a host database on disc storage 48 or on a cloud. When such images have been reconstructed and transferred to storage, data processing server 24 notifies data store server 26. Workstation 12 may be used by an operator to archive the images, produce films, or send the images via a network to other facilities.

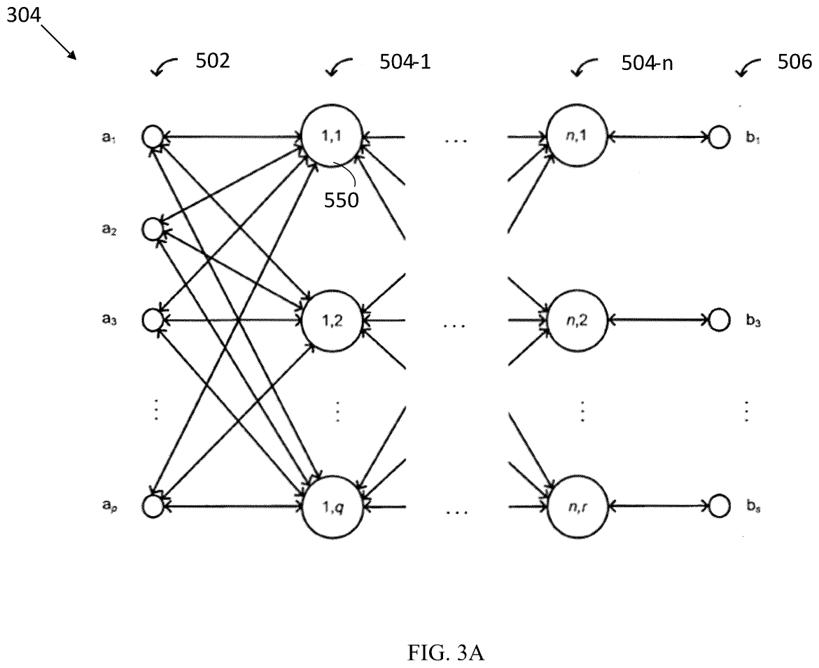

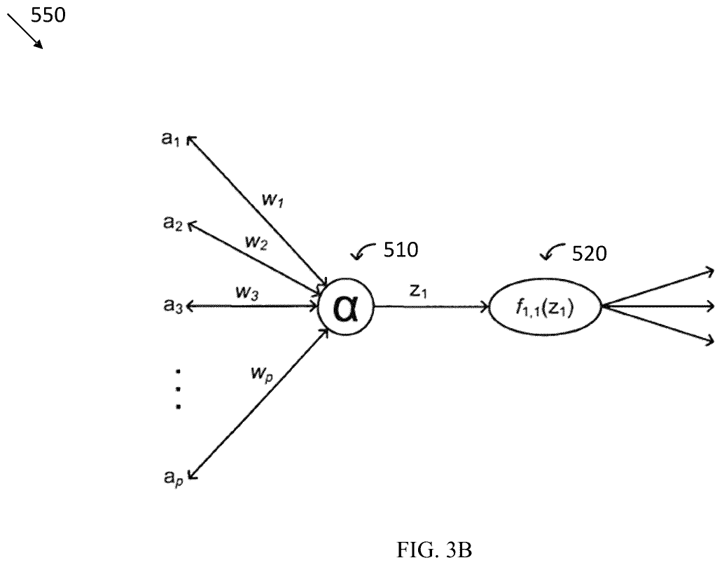

FIG. 3A depicts an example artificial neural network model 304. The example neural network model 304 includes layers of neurons 502, 504-1 to 504-n, and 506, including an input layer 502, one or more hidden layers 504-1 through 504-n, and an output layer 506. Each layer may include any number of neurons, i.e., q, r, and n in FIG. 3A may be any positive integer. It should be understood that neural networks of a different structure and configuration from that depicted in FIG. 3A may be used to achieve the methods and systems described herein.

In the example embodiment, the input layer 502 may receive different input data. For example, the input layer 502 includes a first input a1 representing training images, a second input a2 representing patterns identified in the training images, a third input a3 representing edges of the training images, and so on. The input layer 502 may include thousands or more inputs. In some embodiments, the number of elements used by the neural network model 304 changes during the training process, and some neurons are bypassed or ignored if, for example, during execution of the neural network, they are determined to be of less relevance.

In the example embodiment, each neuron in hidden layer(s) 504-1 through 504-n processes one or more inputs from the input layer 502, and/or one or more outputs from neurons in one of the previous hidden layers, to generate a decision or output. The output layer 506 includes one or more outputs each indicating a label, confidence factor, weight describing the inputs, and/or an output image. In some embodiments, however, outputs of the neural network model 304 are obtained from a hidden layer 504-1 through 504-n in addition to, or in place of, output(s) from the output layer(s) 506.

In the example embodiment, each layer has a discrete, recognizable function with respect to input data. For example, if n is equal to 3, a first layer analyzes the first dimension of the inputs, a second layer the second dimension, and the final layer the third dimension of the inputs. Dimensions may correspond to aspects considered strongly determinative, then those considered of intermediate importance, and finally those of less relevance.

In the example embodiment, the layers are not clearly delineated in terms of the functionality they perform. For example, two or more of hidden layers 504-1 through 504-n may share decisions relating to labeling, with no single layer making an independent decision as to labeling.

FIG. 3B depicts an example embodiment of a neuron 550 that corresponds to the neuron labeled as “1,1” in hidden layer 504-1 of FIG. 3A, according to one embodiment. Each of the inputs to the neuron 550 (e.g., the inputs in the input layer 502 in FIG. 3A) is weighted such that input a1 through ap corresponds to weights w1 through wp as determined during the training process of the neural network model 304.

In the example embodiment, some inputs lack an explicit weight, or have a weight below a threshold. The weights are applied to a function a (labeled by a reference numeral 510), which may be a summation and may produce a value z1 which is input to a function 520, labeled as f1,1 (z1). The function 520 is any suitable linear or non-linear function. As depicted in FIG. 3B, the function 520 produces multiple outputs, which may be provided to neuron(s) of a subsequent layer, or used as an output of the neural network model 304. For example, the outputs may correspond to index values of a list of labels, or may be calculated values used as inputs to subsequent functions.

It should be appreciated that the structure and function of the neural network model 304 and the neuron 550 depicted are for illustration purposes only, and that other suitable configurations exist. For example, the output of any given neuron may depend not only on values determined by past neurons, but also on future neurons.

In the example embodiment, the neural network model 304 may include a convolutional neural network (CNN), a deep learning neural network, a reinforced or reinforcement learning module or program, or a combined learning module or program that learns in two or more fields or areas of interest. Supervised and unsupervised machine learning techniques may be used. In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. The neural network model 304 may be trained using unsupervised machine learning programs. In unsupervised machine learning, the processing element may be required to find its own structure in unlabeled example inputs. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs in the example embodiment may be trained by inputting sample data sets or certain data into the programs, such as images, object statistics, and information. The machine learning programs may use deep learning algorithms that may be primarily focused on pattern recognition, and may be trained after processing multiple examples. The machine learning programs may include Bayesian Program Learning (BPL), voice recognition and synthesis, image or object recognition, optical character recognition, and/or natural language processing-either individually or in combination. The machine learning programs may also include natural language processing, semantic analysis, automatic reasoning, and/or machine learning.

Based upon these analyses, the neural network model 304 in the example embodiment may learn how to identify characteristics and patterns that may then be applied to analyzing image data, model data, and/or other data. For example, the model 304 may learn to identify features in a series of data points.

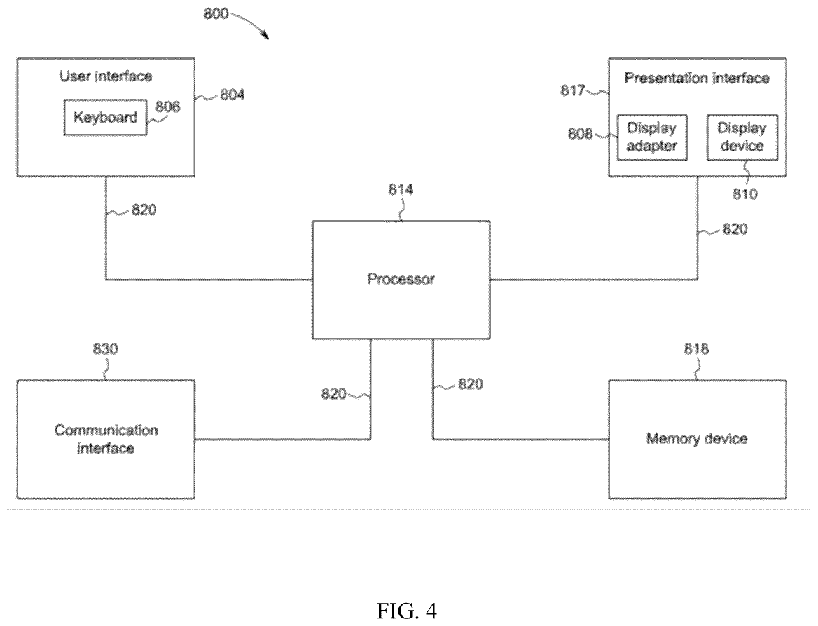

Workstation 12 described herein may be any suitable computing device 800 and software implemented therein. FIG. 4 is a block diagram of an example computing device 800. In the example embodiment, computing device 800 includes a user interface 804 that receives at least one input from a user. User interface 804 may include a keyboard 806 that enables the user to input pertinent information. User interface 804 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the example embodiment, computing device 800 includes a presentation interface 817 that presents information, such as input events and/or validation results, to the user. Presentation interface 817 may also include a display adapter 808 that is coupled to at least one display device 810. More specifically, in the example embodiment, display device 810 may be a visual display device, such as a cathode ray tube (CRT), a liquid crystal display (LCD), a light-emitting diode (LED) display, and/or an “electronic ink” display. Alternatively, presentation interface 817 may include an audio output device (e.g., an audio adapter and/or a speaker) and/or a printer.

Computing device 800 also includes a processor 814 and a memory device 818. Processor 814 is coupled to user interface 804, presentation interface 817, and memory device 818 via a system bus 820. In the example embodiment, processor 814 communicates with the user, such as by prompting the user via presentation interface 817 and/or by receiving user inputs via user interface 804. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are exemplary only, and thus are not intended to limit in any way the definition and/or meaning of the term “processor.”

In the example embodiment, memory device 818 includes one or more devices that enable information, such as executable instructions and/or other data, to be stored and retrieved. Moreover, memory device 818 includes one or more computer readable media, such as, without limitation, dynamic random access memory (DRAM), static random access memory (SRAM), a solid state disk, and/or a hard disk. In the example embodiment, memory device 818 stores, without limitation, application source code, application object code, configuration data, additional input events, application states, assertion statements, validation results, and/or any other type of data. Computing device 800, in the example embodiment, may also include a communication interface 830 that is coupled to processor 814 via system bus 820. Moreover, communication interface 830 is communicatively coupled to data acquisition devices.

In the example embodiment, processor 814 may be programmed by encoding an operation using one or more executable instructions and providing the executable instructions in memory device 818. In the example embodiment, processor 814 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

FIG. 4 is a block diagram of an example computing device 800. Systems and methods described herein may be at least partially implemented with computing device 800. In the example embodiment, computing device 800 includes a user interface 804 that receives at least one input from a user. User interface 804 may include a keyboard 806 that enables the user to input pertinent information. User interface 804 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the example embodiment, computing device 800 includes a presentation interface 817 that presents information, such as input events and/or validation results, to the user. Presentation interface 817 may also include a display adapter 808 that is coupled to at least one display device 810. More specifically, in the example embodiment, display device 810 may be a visual display device, such as a cathode ray tube (CRT), a liquid crystal display (LCD), a light-emitting diode (LED) display, and/or an “electronic ink” display. Alternatively, presentation interface 817 may include an audio output device (e.g., an audio adapter and/or a speaker) and/or a printer.

In the example embodiment, computing device 800 also includes a processor 814 and a memory device 818. Processor 814 is coupled to user interface 804, presentation interface 817, and memory device 818 via a system bus 820. In the example embodiment, processor 814 communicates with the user, such as by prompting the user via presentation interface 817 and/or by receiving user inputs via user interface 804. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are exemplary only, and thus are not intended to limit in any way the definition and/or meaning of the term “processor.”

In the example embodiment, memory device 818 includes one or more devices that enable information, such as executable instructions and/or other data, to be stored and retrieved. Moreover, memory device 818 includes one or more computer readable media, such as, without limitation, dynamic random access memory (DRAM), static random access memory (SRAM), a solid state disk, and/or a hard disk. In the example embodiment, memory device 818 stores, without limitation, application source code, application object code, configuration data, additional input events, application states, assertion statements, validation results, and/or any other type of data. Computing device 800, in the example embodiment, may also include a communication interface 830 that is coupled to processor 814 via system bus 820. Moreover, communication interface 830 is communicatively coupled to data acquisition devices.

In the example embodiment, processor 814 may be programmed by encoding an operation using one or more executable instructions and providing the executable instructions in memory device 818. In the example embodiment, processor 814 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

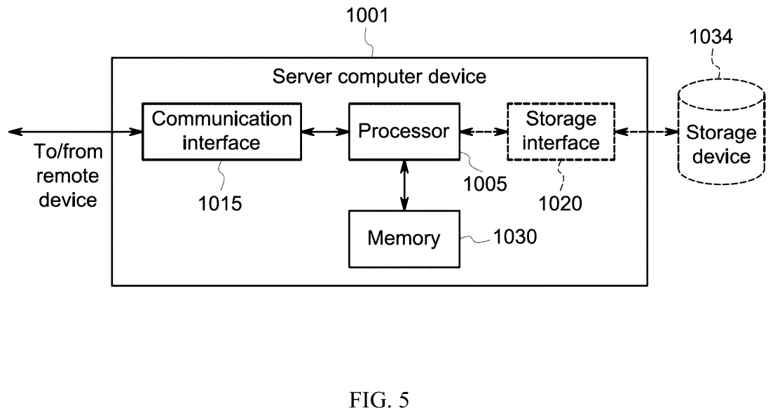

FIG. 5 illustrates an example configuration of a server computer device 1001. Systems and methods described herein may be at least partially implemented with server computer device 1001. Server computer device 1001 also includes a processor 1005 for executing instructions. Instructions may be stored in a memory area 1030, for example. Processor 1005 may include one or more processing units (e.g., in a multi-core configuration).

In the example embodiment, processor 1005 is operatively coupled to a communication interface 1015 such that server computer device 1001 is capable of communicating with a remote device or another server computer device 1001. For example, communication interface 1015 may receive data from workstation 12, via the Internet.

In the example embodiment, processor 1005 may also be operatively coupled to a storage device 1034. Storage device 1034 is any computer-operated hardware suitable for storing and/or retrieving data, such as, but not limited to, wavelength changes, temperatures, and strain. In some embodiments, storage device 1034 is integrated in server computer device 1001. For example, server computer device 1001 may include one or more hard disk drives as storage device 1034. In other embodiments, storage device 1034 is external to server computer device 1001 and may be accessed by a plurality of server computer devices 1001. For example, storage device 1034 may include multiple storage units such as hard disks and/or solid state disks in a redundant array of inexpensive disks (RAID) configuration. Storage device 1034 may include a storage area network (SAN) and/or a network attached storage (NAS) system.

In the example embodiment, processor 1005 is operatively coupled to storage device 1034 via a storage interface 1020. Storage interface 1020 is any component capable of providing processor 1005 with access to storage device 1034. Storage interface 1020 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 1005 with access to storage device 1034.

### Machine Learning & Other Matters

The computer-implemented methods discussed herein may include additional, less, or alternate actions, including those discussed elsewhere herein. The methods may be implemented via one or more local or remote processors, transceivers, and/or sensors (such as processors, transceivers, and/or sensors mounted on mobile devices, or associated with smart infrastructure or remote servers), and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

A processor or a processing element may be trained using supervised or unsupervised machine learning, and the machine learning program may employ a neural network, which may be a convolutional neural network, a deep learning neural network, a reinforced or reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample (e.g., training) data sets or certain data into the programs, such as conversation data of spoken conversations to be analyzed, mobile device data, and/or additional speech data. The machine learning programs may utilize deep learning algorithms that may be primarily focused on pattern recognition, and may be trained after processing multiple examples. The machine learning programs may include Bayesian program learning (BPL), voice recognition and synthesis, image or object recognition, optical character recognition, and/or natural language processing-either individually or in combination. The machine learning programs may also include natural language processing, semantic analysis, automatic reasoning, and/or other types of machine learning, such as deep learning, reinforced learning, or combined learning.

Supervised and unsupervised machine learning techniques may be used. In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. The unsupervised machine learning techniques may include clustering techniques, cluster analysis, anomaly detection techniques, multivariate data analysis, probability techniques, unsupervised quantum learning techniques, associate mining or associate rule mining techniques, and/or the use of neural networks. In some embodiments, semi-supervised learning techniques may be employed. In one embodiment, machine learning techniques may be used to extract data about the conversation, statement, utterance, spoken word, typed word, geolocation data, and/or other data.

### EXAMPLES

### Example 1

Cardiac MRI (CMR) feature tracking (FT) is the most widely used and convenient technique, as it applies post-processing algorithms to standard CMR cine images to assess strain; however, it is less accurate, reproducible and prognostic than strain-dedicated CMR techniques like tagging. The tag lines, deforming along with myocardial movements throughout the cardiac cycle, are crucial for direct measurements of myocardial displacement. As magnetic resonance (MR) tagging has true physical tissue markers, it is an accurate strain CMR method with better prognostic performance than feature tracking (FT) and with high reproducibility for regional strain; however, this method requires the acquisition of additional images dedicated to strain assessment, which adds time to imaging studies, resulting in inefficient clinical workflow and suboptimal utilization of MRI scanners. While accelerated strain-dedicated sequences reduce the scan time, they still require additional acquisitions, breath-holds and complexity for post-processing tagging strain analysis. Therefore, a new strain method with similar performance to MR tagging and the efficiency of FT is needed for accurate detection of regional wall motion abnormalities in various heart diseases.

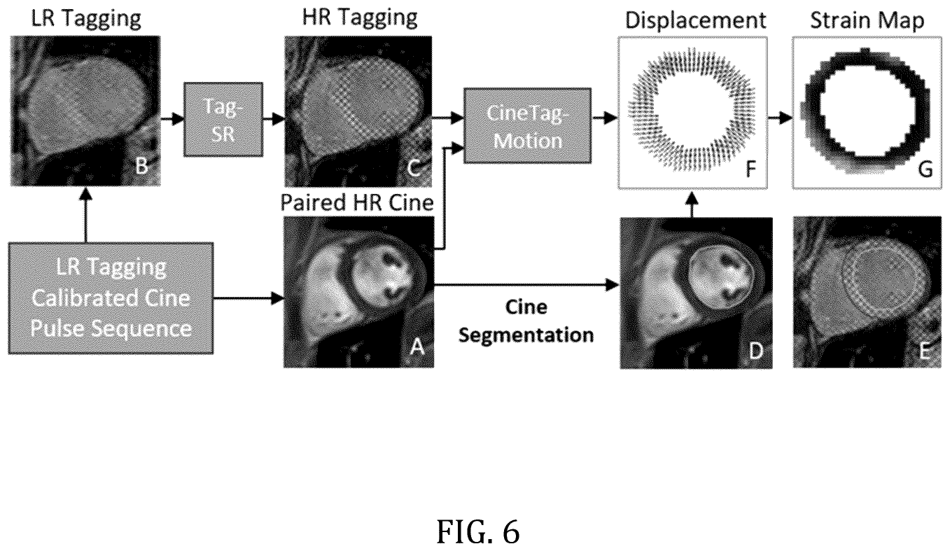

The design is that the low-resolution (LR) tagging calibrated cine MRI provides i) a clinical practical sequence without adding additional breath-holds to routine cine MRI and accurate strain analysis with performance similar to magnetic resonance (MR) tagging and ii) a fully automatic post-processing workflow for clinical use. This approach is to develop a LR tagging calibrated cine MRI sequence which acquires the cine images and paired LR tagging images at the same position, utilize a conditional generative model-based super-resolution (SR) network to enhance tagging image, and track the intramyocardial motion in conjunction with paired cine images. The rationale is that paired cine and tagging images acquired together reflect the same underlying heart motion, with LR tagging providing physical tissue markers and with cine image providing high-resolution (HR) and high-contrast anatomy information to strengthen the tracking power by borrowing information from each other. This is important because regional strain is an indicator of heart function earlier than clinically used left ventricular ejection fraction (LVEF); thus, a fully-automatic and accurate regional strain analysis workflow based on clinical efficient acquisitions are significant and impactful. The overall design is shown in FIG. 6.

LR tagging calibrated cine MRI sequence in a single breath-hold: MR tagging has similar segmented k-space sequence diagram with cine MRI but with an additional preparation SPAtial Modulation of Magnetization (SPAMM) pulse to superimpose tag lines to the myocardium. A LR tagging image is acquired along with cine MRI in a single breath-hold because the additional LR tagging acquisition takes up to 2 heartbeats and therefore may be acquired with the cine images in the same single breath-hold without adding separate sequences, and the paired LR tagging and routine cine images are acquired at the same slice location which provides the opportunity for simplified post-processing tagging analysis by leveraging the matched cine image segmentation. The LR tagging sequence is acquired after the cine MRI sequence to not disturb the original functionality of routine cine sequence. After applying the SPAMM preparation pulse to generate the tag lines, the LR tagging is imaged by acquiring the center 25% phase-encoding lines (rate-4) in k-space with segmented acquisition diagram in 2 heartbeats. For temporal resolution, the LR tagging will have the same cardiac frames as routine cine for simpler matching of subsequent post-processing. To validate the functionality of LR tagging calibrated cine MRI sequence, routine CMR cine sequence and MR tagging sequence at the same slice position are acquired in addition to the hybrid sequence. The cine image quality from the sequence is validated against routine CMR cine, and the MR tagging serves as the ground truth for the LR tagging images and strain analysis.

CineTag-Strain is a fully automatic deep learning (DL) based strain analysis using cine and LR tagging images.

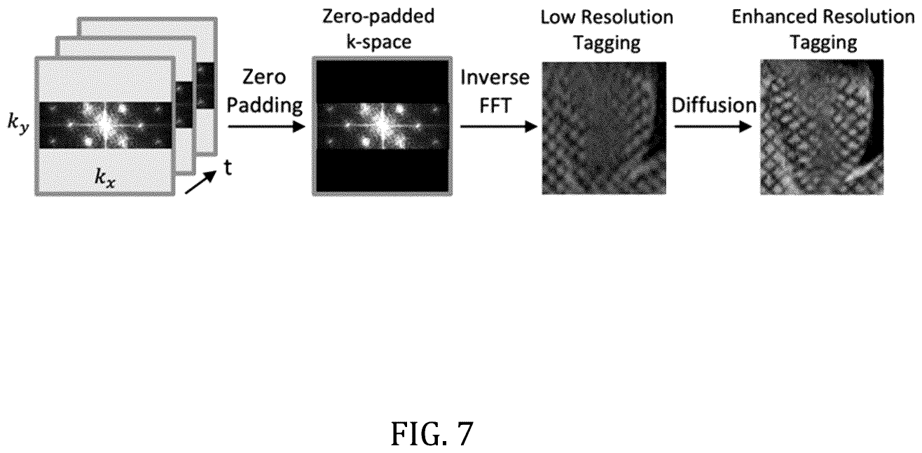

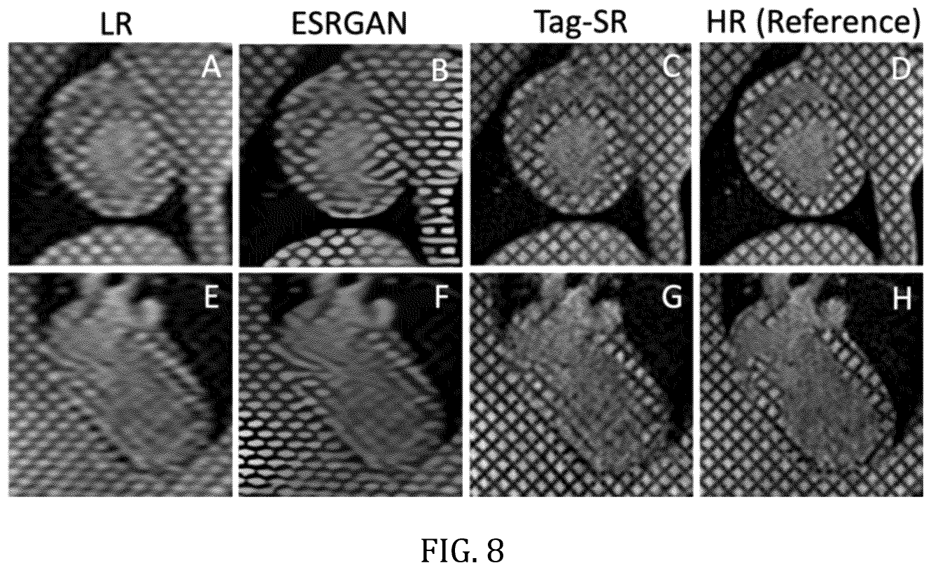

Tag-SR: Tag-SR is a super-resolution (SR) tagging network from LR tagging images. Although LR tagging images contain physical tissue landmarks to be tracked, higher spatial resolution is required to detect fine details features and track the regional wall dysfunction. Generative diffusion models are utilized to build up a SR tagging network, termed CineTag-SR, to generate high resolution (HR) tagging images from LR tagging, and compare with traditional interpolation-based SR method and GAN-based DL network. A generative diffusion model may also be referred to as a generative diffusion machine-learning model. Denoising diffusion probabilistic models (DDPM) and conditional image generation are adapted to perform super-resolution through a stochastic denoising process. The input of the model starts with pure Gaussian noise and a LR tagging image, with the corresponding HR tagging image as the ground truth. The model iteratively refines the noisy output using a U-Net model trained on denoising at various noise levels and generate images with the desired HR tagging data distribution. By conditioning the generation process with LR tagging, the generated enhanced-resolution images is uniquely determined and have the same anatomy similar to the specific LR tagging. The design for Tag-SR is shown in FIG. 7, and the preliminary results are shown in FIG. 8.

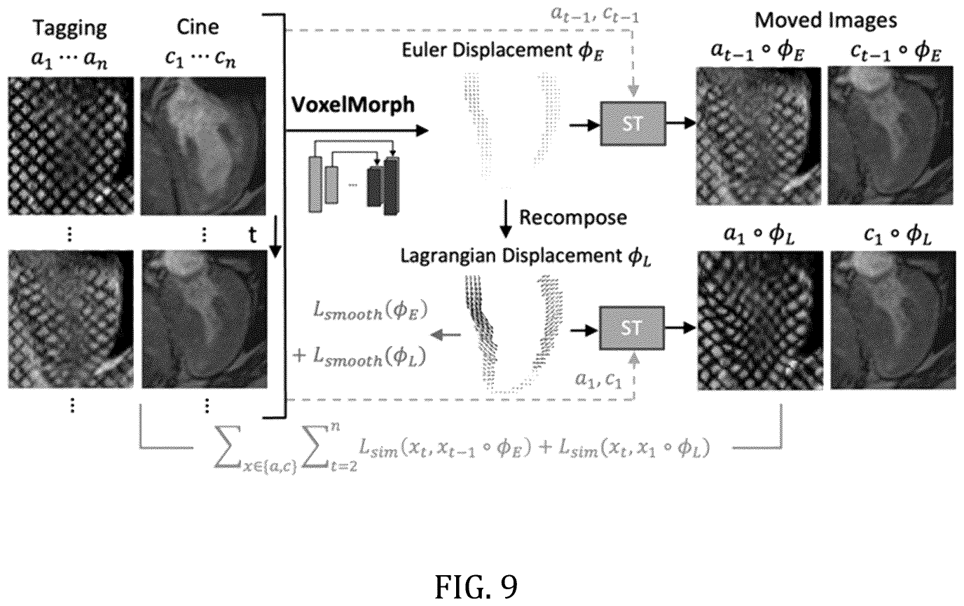

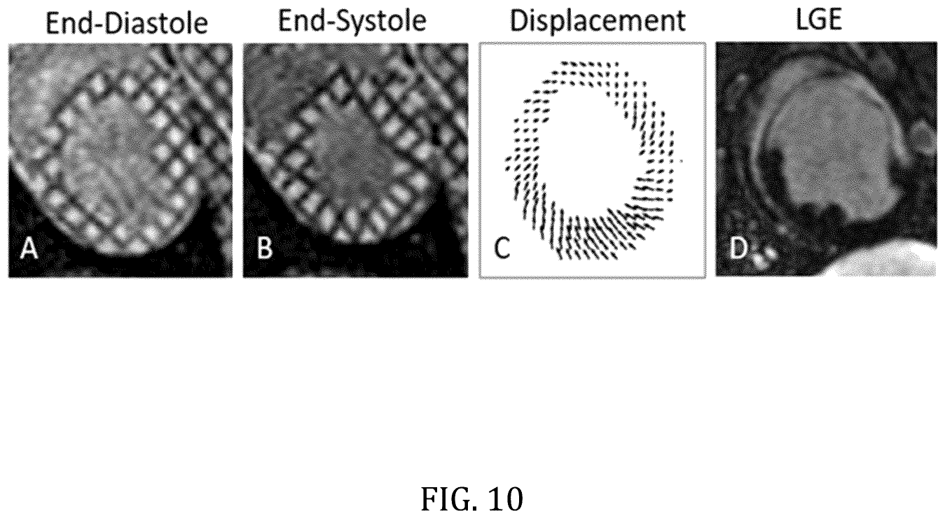

CineTag-Motion: CineTag-Motion is an unsupervised DL method for intramyocardial motion estimation by utilization of both cine and tagging images. A needed process in strain analysis is the accurate measurement of intramyocardial motion, the precursor to strain. The methods utilizes an unsupervised deep learning architecture to estimate the intramyocardial motion based on both HR tagging and HR cine, termed CineTag-Motion. Network Input Design: The input includes multichannel inputs including the paired HR tagging and HR cine benefitting from the matched data acquisition in the same sequence. Compared with solely using of HR tagging images, the paired cine images provide high-resolution anatomy information and similar underlying cardiac motion patterns especially near the endomyocardial and epimyocardial borders and contribute more to the motion tracking when tagging image quality is not perfect. Network Architecture Design includes the VoxelMorph framework to predict the Euler motion field between two successive frames. The model is adept at capturing both small Euler (frame-by-frame) and large Lagrangian (relative to the first frame) displacements, providing a comprehensive analysis of cardiac motion in loss function. The Lagrangian motion field between the reference frame and any other frame is recomposed from the Euler motion field. The loss function is composed of a normalized cross correlation similarity loss between the reference and warped images and a smoothness loss computed by total variation for tagging and cine, respectively. The design for CineTag-Motion is shown in FIG. 9, and the results are shown in FIG. 10.

CineTag-Strain workflow: The CineTag-Strain Workflow includes simplifying the tagging post-processing pipeline by leveraging the well-established cine segmentation. The main challenge of clinical application of MR tagging is the time-consuming and complicated post-processing, which mainly comes from two parts: (a) accurate and efficient motion tracking and image degradation due to tag signal fading, (b) fully automatic and accurate tagging segmentation for myocardial strain analysis. CineTag-SR and CineTag-Motion have provided a solution for (a), and the challenge (b) may also be solved using the simultaneous tagging and cine acquisition design. MR Tagging Segmentation typically requires manual segmentation which is time-consuming and has poor reproducibility for MR tagging. Automated myocardial segmentation provides high-contrast balanced steady state free procession (bSSFP) cine acquisitions, but remains an unsolved issue for MR tagging due to relatively low-contrast myocardial borders. Benefiting from the matched cine and tagging acquisition in the sequence design described herein, the system applies well-developed cine automatic segmentation algorithms to the intermediate cine image and then applies the segmented mask to the paired tagging image. Strain Analysis includes calculating the segmental circumferential strain (Ecc) from short-axis whole-heart tagging and longitudinal strain (Ell) from long-axis (2-, 3-, 4-chamber) tagging with the intramyocardial motion and the myocardial contours. Per-pixel displacement maps and strain maps are computed for visualization of regional wall motions. The overall design is shown FIG. 6.

When the HR tagging image is generated, the tagging image may be utilized to solely compute the cardiac motion and strain analysis. The disclosed methods may also be generalized to conventional tagging which may not have paired cine images. The disclosed systems and methods provide more accurate motion estimation by leveraging both magnitude and phase information and fully-automatic post-processing by leveraging generative diffusion model. Additionally, the fully-automatic post-processing may not only be applied to MR tagging image segmentation, but also any type of cardiac images.

A physics-guided cardiac tagging MRI motion estimation using an unsupervised neural network is also provided. Routine clinical use of myocardial tagging is limited due to the time-intensive post-processing required. An Integrated Magnitude-Phase Approach for Cardiac Tagging (IMPACT) MRI utilizes an unsupervised neural network to perform the post-processing (IMPACTNet). By integration of magnitude and phase image data, IMPACTNet enhances both the accuracy and automation of tagging MRI post-processing, improving motion estimation and clinical utility. This approach, synergizing MR physics with advanced computer vision techniques outperforms traditional single-source tracking methods. The unsupervised learning design effectively overcomes the challenge of limited tagging datasets with ground truth motion, making it a more accurate, reliable, and automated solution for cardiac motion analysis.

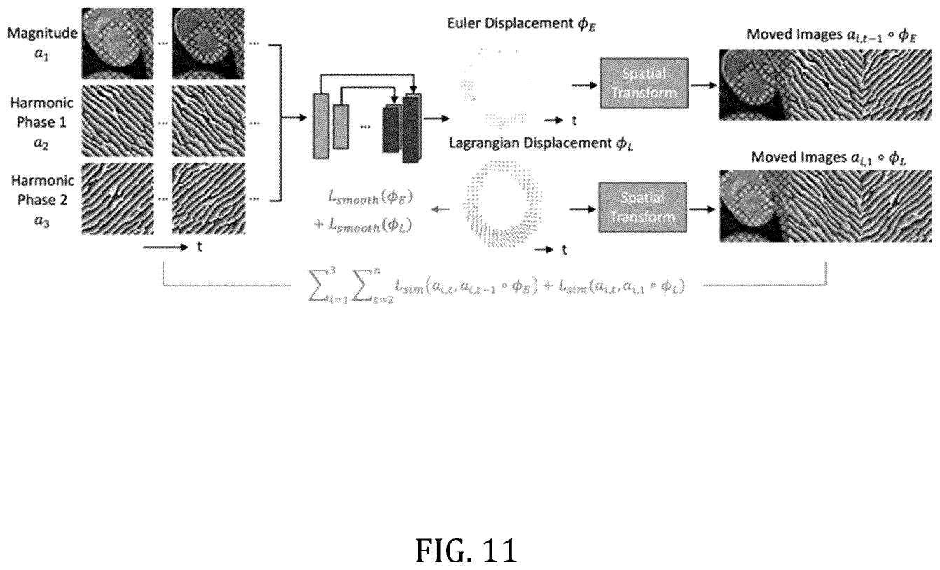

Cardiac Tagging MRI is the standard for assessing regional myocardial deformation and cardiac strain, but it faces challenges with traditional post-processing techniques. These range from computationally intense and complex physics-based methods like Harmonic Phase (HARP) and sine-wave modeling (SinMod) to faster but less accurate magnitude-based methods like optical flow, which struggles with tag line decay and artifacts. IMPACTNet addresses these challenges by leveraging the strengths of both phase and magnitude information (FIG. 11). IMPACTNet employs the consistent phase principle in phase images for precise motion tracking, incorporated by the feature tracking of magnitude images. This is further enhanced by the ability of convolutional neural network (CNN) and transformer to extract richer features from multiple scales and long-term dependency from multiple temporal frames. Employing deep learning, IMPACTNet processes magnitude and phase signals as multi-channel inputs simultaneously (FIG. 11). This simultaneous utilization allows for integrating information from both sources in CNN and transformer learning, which enhances accuracy and improves computational efficiency. The model is adept at capturing both small Euler (frame-by-frame) and large Lagrangian (relative to the first frame) displacements, offering a comprehensive analysis of cardiac motion in loss function. Faced with the scarcity of ground truth data, IMPACTNet adopts an unsupervised learning strategy. It updates model parameters using similarity loss, calculated across both spatial and temporal dimensions of displacement fields. This method avoids motion tracking relying on potentially suboptimal estimation based on traditional methods. The network loss ensures that images warped by the estimated motion at any given time align closely with the actual images, enhancing the accuracy of motion estimation. The tagging dataset includes data from 12 healthy volunteers and 100 patients with both short-axis and long-axis views acquires from June 2022 to November 2023, enables the development of IMPACTNet.

Additional embodiments include a fully automated pipeline for cardiac tagging MRI, enabling precise, automated derivation of longitudinal and circumferential strain. Integrating deep learning with MR physics, it promises to expand research and clinical utility for tagging MRI.

Additionally, systems and methods for standardized automatic cardiac MR Image Segmentation Using Generative AI are provided. One of the challenges of cardiac MRI (CMR) is that automated myocardial segmentation is well developed for high-contrast balanced Steady State Free Precession (bSSFP) cine acquisitions, but remains an unsolved issue for other types of acquisitions in which tissue contrast is lower. Manual segmentation is tedious, time-consuming, and has poor reproducibility. The disclosed method uses a generative AI model to create a high-contrast pseudo-bSSFP cine image set from any of a variety of other CMR sequences (e.g., tagging and perfusion). By generating a pseudo-bSSFP cine series with anatomical structure based on the input sequence images, a bSSFP cine automatic segmentation algorithm may be applied to the intermediate pseudo-bSSFP cine image. A segmented mask may then be applied to the original image. The conditional generative model provides effective cine conversion without paired cine and other sequences but preserves the myocardial structure by adding the anatomy condition during inference. This model efficiently handles sequences such as tagging or long-axis images where automatic analysis is restricted by limited data and low contrast between blood pool and myocardial regions, and significantly diminishes the need for extensive curated datasets.

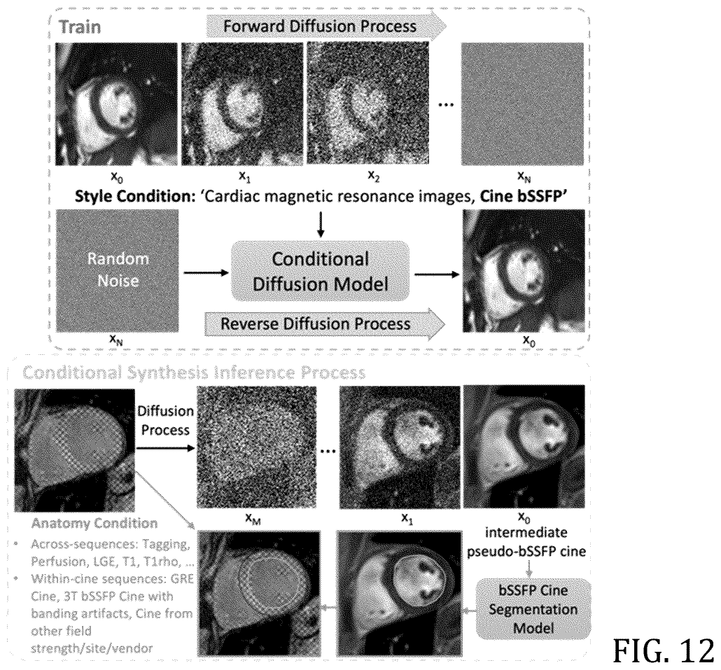

The diffusion model, a deep learning approach, has shown superior accuracy in generating semantic elements and object shapes over existing methods in computer vision. Rather than randomly generating a bSSFP cine image, the model generates pseudo bSSFP cine images from a range of sequences while maintaining precise anatomical structures as shown in FIG. 12. By preserving semantic elements and anatomical accuracy, this approach bridges the segmentation gap across various sequences. This method generates aligned cine images with conditional images from other sequences during inference (FIG. 12, Conditional Synthesis Inference process). By ensuring these generated cine images retain the same myocardial structure, the system may apply segmentation directly to the original sequences. This process effectively streamlines myocardial structure generation, reducing the need for paired datasets or manual contours. The ‘cine’ style is seamlessly maintained throughout both training and inference, achieved through textual descriptions ‘cine’.

The technique utilizes a conditional generative AI model to leverage inherent anatomical similarities across various CMR sequences. It is crucial for achieving standardized analysis and addresses the challenges of limited labeled segmentation datasets across different CMR sequences, scanners, and magnetic field strengths. In addition to segmentation, the disclosed model may also be applied to generate corresponding high-quality image to mitigate banding artifacts, enhance resolution of real-time cine image or contrast of 3D bSSFP cine MRI.

Example embodiments of systems and methods for cardiac strain measurement are described above in detail. The systems and methods are not limited to the specific embodiments described herein but, rather, components of the systems and/or operations of the methods may be utilized independently and separately from other components and/or operations described herein. Further, the described components and/or operations may also be defined in, or used in combination with, other systems, methods, and/or devices, and are not limited to practice with only the systems described herein.

### Example 2

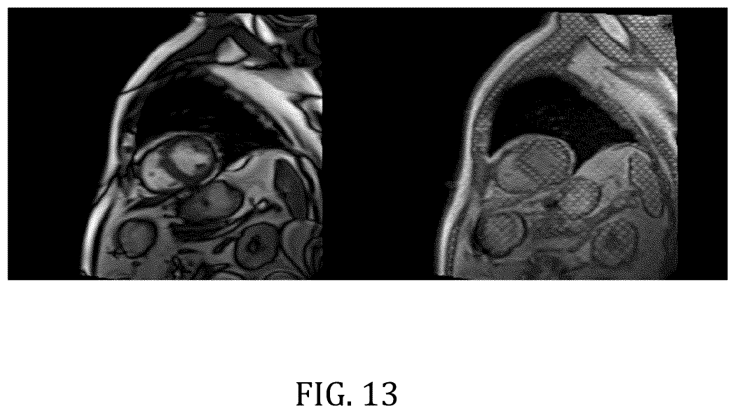

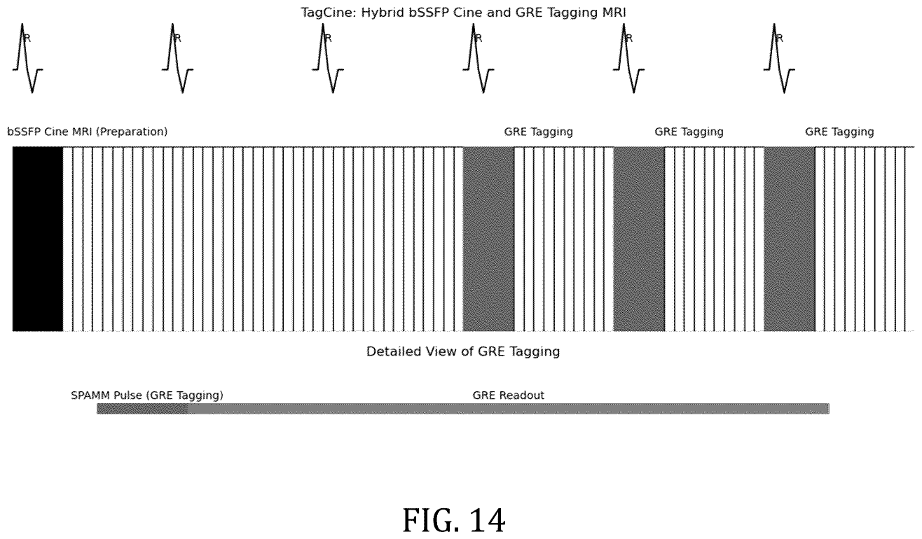

FIGS. 13 and 14 show an example acquisition of cine images and tagging images for the systems and methods described herein. FIG. 13 shows example images acquired. The left image is a cine MRI, while the right image is a tagging MRI, both acquired in the same scan within a single breath-hold over just nine heartbeats. This MRI sequence provides complementary imaging modalities for a shared objective: measuring myocardial motion and strain while mitigating each method's limitations. Cine MRI enables straightforward feature tracking but may lack precision, whereas tagging MRI provides physical markers for more accurate motion analysis. Additionally, cine MRI offers better contrast, facilitating easier myocardial border tracking and segmentation. This matched scan approach presents a promising solution for the automated analysis of heart motion using MRI.

Referring to FIG. 14, an example pulse sequence used in acquiring the images is provided. The sequence design begins with a bSSFP cine acquisition, followed by GRE-based tagging MRI. This approach leverages the advantages of both techniques: bSSFP cine provides high contrast for easy myocardial segmentation and feature tracking, while GRE tagging MRI introduces physical markers for precise myocardial strain analysis. By acquiring both sequences in a single breath-hold, this design ensures spatial and temporal alignment, facilitating automated analysis and enhancing motion tracking accuracy. bSSFP and GRE-based acquisitions are shown for illustration purposes only. Other pulse sequences may be used to acquire cine images and/or tagging images.

An example technical effect of the methods, systems, and apparatus described herein includes at least one of: (a) increasing accuracy in cardiac strain maps using high-resolution tagging images derived from low-resolution tagging images, without acquiring high-resolution tagging images and resulted increase in scan time, breath-holds, and/or complexity in post-processing, (b) increasing speed in myocardial segmentation by generating pseudo-bSSFP cine images without acquisition of bSSFP cine images, (c) estimating myocardial motion using a neural network model trained via unsupervised training, thereby avoiding the challenges from limited availability of training datasets that have ground truth of motion, or (d) increasing accuracy in cardiac strain maps using cine images and tagging images.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” and/or “substantially” as applied to a particular value of a range applies to both values, and unless otherwise dependent on the precision of the instrument measuring the value, may indicate +/−10% of the stated value(s).

As used herein, an element or step recited in the singular and proceeded with the word “a” or “an” should be understood as not excluding plural elements or steps, unless such exclusion is explicitly recited. Furthermore, references to “example” or “one example” of the present disclosure are not intended to be interpreted as excluding the existence of additional examples that also incorporate the recited features. Further, to the extent that terms “includes,” “including,” “has,” “contains,” and variants thereof are used herein, such terms are intended to be inclusive in a manner similar to the term “comprises” as an open transition word without precluding any additional or other elements.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method for analyzing myocardial strain in a subject using magnetic resonance imaging (MRI), the method comprising:
acquiring, via a magnetic resonance (MR) system, cine images and low-resolution tagging images of a cardiac region of a subject within a single breath-hold of the subject, the cine images having a first resolution, the low-resolution tagging images having a second resolution lower than the first resolution;
deriving high-resolution tagging images based on the low-resolution tagging images, the high-resolution tagging images having a resolution higher than the second resolution;
estimating intramyocardial motion based on the high-resolution tagging images and/or the cine images;
generating myocardial strain maps based on the intramyocardial motion; and
outputting the myocardial strain maps.

2. The method of claim 1, wherein deriving the high-resolution tagging images further comprises:
deriving the high-resolution tagging images using a first neural network model, wherein the first neural network model is trained with a pair of pristine images and crude images, wherein the pristine images are the crude images with noise reduced and/or having a resolution higher than a resolution of the crude images, and target output images of the first neural network model are the pristine images.

3. The method of claim 2, wherein deriving the high-resolution tagging images further comprises:
deriving, using the first neural network model including a generative diffusion machine-learning model, the high-resolution tagging images, wherein the generative diffusion machine-learning model is trained by:
inputting noise images and crude images; and
using pristine images as ground truth.

4. The method of claim 3, wherein deriving the high-resolution tagging images further comprises:
deriving, using the generative diffusion machine-learning model, by conditioning the generative diffusion machine-learning model with the low-resolution tagging images.

5. The method of claim 2, wherein the pair of pristine images and the crude images are generated by:
applying a Fourier transform to the pristine images to generate pristine k-space data;
under-sampling the pristine k-space data into under-sampled k-space data; and
applying an inverse Fourier transform to the under-sampled k-space data to generate the crude images.

6. The method of claim 1, wherein estimating the intramyocardial motion further comprises:
estimating the intramyocardial motion using a second neural network model, wherein the second neural network model is trained via unsupervised training with pairs of training cine images and training high-resolution tagging images, the training high-resolution tagging images having a resolution higher than the second resolution, wherein the pairs of the training cine images and the training high-resolution tagging images are input into the second neural network model during the unsupervised training.

7. The method of claim 6, wherein estimating the intramyocardial motion further comprises:
estimating the intramyocardial motion using the second neural network model, the second neural network model configured to predict the intramyocardial motion including frame-by-frame displacements and displacements from a first frame in the high-resolution tagging images and/or the cine images.

8. The method of claim 6, wherein the second neural network model is trained using a loss function including a similarity loss and/or a smoothness loss.

9. The method of claim 1, wherein estimating the intramyocardial motion further comprises:
estimating the intramyocardial motion by:
inputting magnitude images and/or phase images of the high-resolution tagging images and/or magnitude images of the cine images into a third neural network model, the third neural network model having a plurality of input channels.

10. The method of claim 1, wherein generating the myocardial strain maps further comprises:
deriving pseudo-balanced steady state free precession (bSSFP) cine images based on the cine images and/or the high-resolution tagging images, the pseudo-bSSFP cine images having a contrast resembling a contrast of images acquired by a bSSFP MR pulse sequence.

11. The method of claim 10, wherein deriving the pseudo-bSSFP cine images further comprises:
deriving, using a generative diffusion machine learning model, the pseudo-bSSFP cine images by conditioning the generative diffusion machine learning model with the cine images and/or the high-resolution tagging images.

12. The method of claim 10, wherein generating the myocardial strain maps further comprises:
generating masks based on the pseudo-bSSFP cine images;
generating the myocardial strain maps by:
applying generated masks to images of the cine images and/or the high-resolution tagging images to generate myocardial contours; and
generating the myocardial strain maps based on the intramyocardial motion and the myocardial contours.

13. The method of claim 1, wherein acquiring the cine images and the low-resolution tagging images further comprises:
acquiring the cine images and the low-resolution tagging images by applying an MR pulse sequence including a cine acquisition and a tagging acquisition.

14. A computer-implemented method for analyzing myocardial strain in a subject using magnetic resonance imaging (MRI), the method comprising:
receiving low-resolution tagging images of a cardiac region of a subject, the low-resolution tagging images acquired via a magnetic resonance system within a single breath-hold of the subject;
deriving high-resolution tagging images based on the low-resolution tagging images, the high-resolution tagging images having a resolution higher than the low-resolution tagging images;
estimating intramyocardial motion based on the high-resolution tagging images;
generating myocardial strain maps based on the intramyocardial motion; and
outputting the myocardial strain maps.

15. The method of claim 14, wherein deriving the high-resolution tagging images further comprises:
deriving the high-resolution tagging images using a first neural network model, wherein the first neural network model is trained with a pair of pristine images and crude images, wherein the pristine images are the crude images with noise reduced and/or having a resolution higher than a resolution of the crude images, and target output images of the first neural network model are the pristine images.

16. The method of claim 15, wherein deriving the high-resolution tagging images further comprises:
deriving, using the first neural network model including a generative diffusion machine-learning model, the high-resolution tagging images by conditioning the generative diffusion machine-learning model with the low-resolution tagging images, wherein the generative diffusion machine-learning model is trained by:
inputting noise images and crude images; and
using pristine images as ground truth.

17. The method of claim 15, wherein the pair of pristine images and the crude images are generated by:
applying a Fourier transform to the pristine images to generate pristine k-space data;
under-sampling the pristine k-space data into under-sampled k-space data; and
applying an inverse Fourier transform to the under-sampled k-space data to generate the crude images.

18. The method of claim 14, wherein estimating the intramyocardial motion further comprises:
estimating the intramyocardial motion by:
inputting magnitude images and/or phase images of the high-resolution tagging images into a third neural network model, the third neural network model having a plurality of input channels.

19. The method of claim 14, wherein generating the myocardial strain maps further comprises:
deriving pseudo-balanced steady state free precession (bSSFP) cine images based on the high-resolution tagging images, the pseudo-bSSFP cine images having a contrast resembling a contrast of images acquired by a bSSFP MR pulse sequence.

20. The method of claim 19, wherein deriving the pseudo-bSSFP cine images further comprises:
deriving, using a generative diffusion machine learning model, the pseudo-bSSFP cine images by conditioning the generative diffusion machine learning model with the high-resolution tagging images.
